(19) United States
(12) Patent Application Publication
(10) Pub. No.: US 2024/0199514 A1
(43) Pub. Date: Jun. 20, 2024

(54) A PROCESS FOR THE CONVERSION OF GLYCEROL TO PROPANOLS

(71) Applicants: ENI S.P.A., Roma (RM) (IT); BASF SE, Ludwigshafen am Rhein (DE)

(72) Inventors: Giacomo FILIPPINI, San Donato Milanese (MI) (IT); Gianluca FIORI, San Donato Milanese (MI) (IT); Thomas PASINI, San Donato Milanese (MI) (IT); Laura Annamaria PELLEGRINI, Alzano Lombardo (BG) (IT); Steffen MADER, Mannheim (DE); Stephanie Sybille LINKE, Mannheim (DE); Stefan KOTREL, Weinheim (DE); Thomas HEIDEMANN, Viernheim (DE); Dagmar Pascale KUNSMANN-KEITEL, Limburgerhof (DE)

(21) Appl. No.: 18/573,216

(22) PCT Filed: Jun. 27, 2022
(86) PCT No.: PCT/IB2022/055948

§ 371 Date: Dec. 21, 2023

(30) Foreign Application Priority Data
Jun. 28, 2021 (IT) 102021000016859

(51) Int. Cl. C07C 29/60; C07C 29/84; C07C 31/08; C07C 31/10

(52) U.S. Cl. CPC C07C 29/60; C07C 29/84; C07C 31/08; C07C 31/10

(57) ABSTRACT

A process to produce propanol and iso-propanol (bio-propanol), a biocomponent for gasoline relates to the conversion of bio-glycerin to bio-propanol and bio-iso-propanol.

In particular, a process is related for the conversion of glycerin, in particular glycerin from renewable sources, to propanols, the process including the following steps:
(a) hydrogenating a glycerin phase with a Co—Cu—Mn—Mo based hydrogenation catalyst to give an effluent containing water and an organic mixture of more than 40 wt % of a mixture of ethanol, 1-propanol and 2-propanol and the rest being unreacted propanediols and glycerin, with traces of ethylene glycol;
(b) separating by mainly distillation the ethanol, 1-propanol and 2-propanol mixture from the other components in the effluent of step a); and (c)
optionally, recycling all or part of the unreacted propandiols and glycerin deriving from steps a) and/or b) to the hydrogenation step a).

### CROSS-REFERENCE TO RELATED APPLICATIONS

This application is a 35 U.S.C. § 371 National Stage patent application of PCT/IB2022/055948, filed on 27 Jun. 2022, which claims the benefit of Italian patent application 102021000016859, filed on 28 Jun. 2021, the disclosures of which are incorporated herein by reference in their entirety.

### TECHNICAL FIELD

The present disclosure relates to a process to produce propanol and iso-propanol (bio-propanol), a biocomponent for gasoline. The disclosure particularly relates to the conversion of bio-glycerin to bio-propanol and bio-iso-propanol.

### BACKGROUND

It is known that the emissions produced by burning fuels of fossil origin containing carbon dioxide (CO₂), carbon monoxide (CO), nitrogen oxides (NOₓ), sulfur oxides (SOₓ), unburned hydrocarbons (HC), volatile organic compounds and particulate matter (PM), are the cause of environmental problems, such as the production of ozone, the greenhouse effect (in the case of nitrogen and carbon oxides), acid rain (in the case of sulfur and nitrogen oxides).

The constant increase in the consumption of fuels for transportation and the increasingly greater sensitivity towards the environment, along with an increasingly stringent international framework of legislation in relation to pollutant emissions and greenhouse gases, have led to the constantly greater importance of processes to allow fuels to be obtained from renewable sources, so-called bio-fuels.

In particular, with the adhesion to the Kyoto Protocol, the European Union has issued a series of directives, known as the “20120120 package”, which set out the aim of reducing global warming due to human activity. The revision of the RED Directive, known as the “ILUC Directive” encourages the use of “advanced” bio-fuels waste, (i.e. deriving from municipal algae, waste effluents containing crude glycerin, lignocellulosic biomass, etc.).

A bio-additive commonly used in biofuels is ethanol. However, the use of ethanol mixed with gasoline is not free from drawbacks. In fact, ethanol is hygroscopic, miscible with water and immiscible with hydrocarbon mixtures in a wide temperature range. Furthermore, ethanol is characterized by low calorific power, and the high latent heat of vaporization can cause problems for cold starts. Additionally, ethanol can form azeotropes with light hydrocarbons causing an increase in the volatility of the fuel containing it.

Propanols have the same positive characteristics as ethanol with reference to its ignition qualities and calorific power, but they have a higher energy density (+12%), lower volatility, and lower latent heat of vaporization (−8%). A good source of propanols can be in principle glycerin.

Glycerin (glycerol or 1, 2, 3-propanetriol) is a polyol of great industrial interest, either used as such or as an intermediate for the production of cosmetics, drugs/nutraceuticals and for the animal feed industry. It is mainly obtained from triglycerides—the main components of animal and plants fats and oils—as a co-product in the reactions of saponification, hydrolysis and transesterification taking place in oleo-chemical plants and in the production process of biodiesel. In particular, glycerol obtained as a co-product in biodiesel synthesis is estimated to be around a few million tons per year.

Several possibilities are described in literature for the valorization of glycerol as a raw material, being the hydrogenolysis of C—O bonds with formation of propanediols, propanols and propane highly interesting for obtaining gasoline and biofuels additives. However, such process is highly complex since the reaction mechanism is strongly dependent on both reaction conditions and catalyst used.

Normally, the reaction is carried out in liquid phase using batch systems working at high hydrogen pressures (autoclaves) using diluted mixtures of glycerol in water, in order to maximize the solubility of the molecular hydrogen in the liquid phase, therefore favoring the mass transfer and avoiding limitations related to the low diffusivity of hydrogen (diffusive regime).

U.S. Pat. No. 5,616,817 discloses a method of producing, as a main components, 1,2-propanediol by catalytic hydrogenation of glycerol at high temperature and pressure, which implies using glycerol with a water content of up to 20% by weight and a catalyst containing from 40 to 70% by weight of cobalt, from 10 to 20% by weight of copper, from 0 to 10% by weight of manganese and from 0 to 10% by weight of molybdenum, and which may additionally contain inorganic polyacids and/or heteropolyacids up to 10% by weight.

The synthesis of 1,2-propanediol has already been brought to industrialization using various catalysts such as chromite copper. It is also well-known that such reaction is promoted by transition metal-based catalysts (especially Cu, Ni and Co based, for example Cu/Sio₂, Cu/Al₂O₃, Ni Raney, cobalt-aluminum alloys), at temperatures between 120 and 220° C. The above-mentioned catalysts are generally selective to 1,2-propanediol, as they are unable to promote consecutive reactions to form propanols.

Furthermore, several monometallic noble metal nanoparticle-based catalysts, especially Ru, Pt and Rh on various supports (coal, TiO₂, SiO₂, Al₂O₃), have been tested to produce propanols in a temperature range between 170 and 220° C. Among these, Rh and Ru based catalysts have proved to be the most active for hydrogenolysis, leading 10 to high glycerol conversions and to the formation of mixtures rich in 1,2-propanediol and hydrogenolysis consecutive reactions products (the latter with low yields and selectivity). Nevertheless, noble metals catalysts used as such, without a co-catalyst, do not lead to significant advantages compared to the transition metal-based systems. In order to optimize the catalytic activity and to enhance consecutive reactions, co-catalysts are often added to noble metal-based catalysts.

It has been verified that the addition of acid co-catalysts (Amberlyst resins, sulfonated zirconia, acid zeolites, heteropolyacids) to Ru and Rh-based catalysts leads to an improvement of the catalytic performances in terms of glycerol conversion and 1,2-propanediol yields, while increasing to some extent the formation of propanols under more severe reaction conditions (i.e. higher temperature and hydrogen pressure).

Relevant literature relating to the above methods are: Miyazawa, T., Koso, S., Kunimori, K., Tomishige, K., Development of a Ru/C Catalyst for Glycerol Hydrogenolysis in Combination with an Ion-Exchange Resin, Appl. Catal. A Gen. 2007, 318 (3), 244−251; Balaraju, M., Rekha, V., Prasad, P. S. S., Devi, B. L. A. P., Prasad, R. B. N., Lingaiah, N., Influence of Solid Acids as Co-Catalysts on Glycerol Hydrogenolysis to Propylene Glycol over Ru/C Catalysts, Appl. Catal. A Gen. 2009, 354 (1−2), 82-87. The addition of basic co-catalysts (LiOH, NaOH, CaO) is much less frequent and often leads to an increase in glycerol conversions, but also to worse selectivity in PDO, promoting the breakdown of C—C bonds with the formation of ethylene glycol (EG) and lactic acid esters (Maris, E. P., Ketchie, W. C., Murayama, M., Davis, R. J., Glycerol Hydrogenolysis on Carbon-Supported PtRu and AuRu Bimetallic Catalysts, J. Catal. 2007, 251 (2), 281−294; Maris, E. P., Davis, R. J., Hydrogenolysis of Glycerol over Carbon-Supported Ru and Pt Catalysts, J. Catal. 2007, 249 (2), 328−337). The latter are probably originated by the Cannizzaro reaction, occurring from 2-hydroxypropionaldehyde—obtained from the dehydrogenation of 1,2-propanediol—with formation and subsequent esterification of lactic acid.

Propanols can be produced using noble metals-based catalysts, modified by dopants and promoters, especially metal oxides (groups 4−7) (Shinmi, Y., Koso, S., Kubota, T., Nakagawa, Y., Tomishige, K., Modification of Rh/SiO₂ catalyst for the Hydrogenolysis of Glycerol in Water, Appl. Catal. B Environ. 2010, 94 (3−4), 318−326), however the cost of the catalyst for this reaction is very high.

Dopants and promoters are capable to modify the electronic properties, therefore the reagents adsorption capacities, of the metallic active phase nanoparticles. In fact, it is well-known that the inclusion of rhenium oxide (ReOₓ) greatly increases the catalytic activity of the nanoparticles of Rh (Shinmi, Y., Koso, S., Kubota, T., Nakagawa, Y., Tomishige, K., Modification of Rh/SiO₂catalyst for the Hydrogenolysis of Glycerol in Water, Appl. Catal. B Environ. 2010, 94 (3−4), 318−326; Tomishige, K., Nakagawa, Y., Tamura, M., Selective Hydrogenolysis and Hydrogenation Using Metal Catalysts Directly Modified with Metal Oxide Species, Green Chem. 2017, 19 (13), 2876−2924), Pd (Ota, N., Tamura, M., Nakagawa, Y., Okumura, K., Tomishige, K., Performance, Structure, and Mechanism of ReOₓ-Pd/CeO₂ Catalyst for Simultaneous Removal of Vicinal OH Groups with H₂, ACS Catal. 2016, 6 (5), 3213−3226) and Ru (Tamura, M., Amada, Y., Liu, S., Yuan, Z., Nakagawa, Y., Tomishige, K., Promoting Effect of Ru on Ir-ReOₓ/SiO₂ catalyst in Hydrogenolysis of Glycerol, J. Mol. Catal. A Chem. 2014, 388−389, 177−187), promoting the selective formation of 1,3-propanediol.

While propanediol is a good starting material in several industrial applications, it is not apt to be used as an additive for biofuels, as reported in the European Standards for Gasoline EN 228.

There is therefore the need to provide a process for converting glycerin into propanols with a high degree of conversion and selectivity and with a low content of propanediols.

### SUMMARY

The present disclosure provides a hydrogenation process to give propanols starting from glycerin, specifically to give bio-propanol and bio-iso-propanol starting from bio-glycerin, as defined in the appended claims, whose recitations are to be considered part of the present description for the requirement of sufficiency of disclosure.

In particular, the present disclosure relates to a process for the conversion of glycerin, in particular glycerin from renewable sources, to propanols, the process comprising the following steps:
a) Hydrogenating a glycerin phase with a Co—Cu—Mn—Mo based hydrogenation catalyst to give an effluent containing water and an organic mixture of more than 40 wt % of a mixture of ethanol, 1-propanol and 2-propanol and the rest being unreacted propanediols and glycerin, with traces of ethylene glycol;
b) Separating by mainly distillation the ethanol, 1-propanol and 2-propanol mixture from the other components in the effluent of step a);
c) Optionally, recycling all or part of the unreacted propandiols and glycerine deriving from steps a) and/or b) to the hydrogenation step a).

The disclosure also provides a plant for actuating the process of the disclosure, comprising:
at least one hydrogenation reactor filled with the hydrogenation catalyst;
at least one first distillation column configured for separating the mixture composed by propanol, iso-propanol, ethanol and water coming from the at least one hydrogenation reactor from the other reactor effluent components, wherein the heaviest components, mainly 1,2 propanediol, 1,3 propanediol and ethylene glycol along with unreacted glycerol, are removed from the bottom;
at least one second distillation column configured for extractive distillation to separate water and ethylene glycol as a an entrainer solvent from the bottom and high purity propanol, iso-propanol and ethanol from the top;
optionally, at least one third distillation column configured to separated ethylene glycol from the bottom and water from the top.

Further characteristics and advantages of the present disclosure will become clear from the following detailed description.

### BRIEF DESCRIPTION OF THE DRAWINGS

FIG. 1 shows the flow chart of a simplified embodiment of a plant for the hydrogenation of glycerol according to the disclosure;

FIG. 2 shows the flow chart of another embodiment of a plant for the hydrogenation of glycerol and for the subsequent recovering of an alcohol fraction according to the disclosure;

FIG. 3 shows the flow chart of a third embodiment of the hydrogenation section of a plant for the hydrogenation of glycerol according to the disclosure;

FIG. 4 shows the flow chart of the distillation section of the plant of FIG. 3;

FIG. 5 shows the flow chart of a variant of the plant of FIG. 3 for the hydrogenation of glycerol according to the disclosure; and

FIG. 6 shows the flow chart of a variant of the plant of FIG. 4.

### DETAILED DESCRIPTION OF THE DRAWINGS

For the purposes of the present description and following claims, the definitions of the numeric ranges always include the extremes unless specified otherwise.

In the description of the embodiments of the present disclosure, the use of the terms “comprising” and “containing” indicates that the options described, for example regarding the steps of a method or of a process or the components of a product or of a device, are not necessarily all-inclusive. It is however important to note that the present application also relates to the embodiments in which the term “comprising” in relation to the options described, e.g. regarding the steps of a method or of a device, must be interpreted as “which essentially consists of” or “which consists of”, even if this is not explicitly stated.

For the purposes of the present disclosure, the term “fuel” means “diesel or gasoline”.

For the purposes of the present disclosure, the term “diesel” means a mixture mainly comprised of hydrocarbons such as paraffins, aromatic hydrocarbons and naphthenes, typically having 9 to 30 carbon atoms, which can be used as fuel. Generally, the distillation temperature of diesel is comprised between 180° C. and 450° ° C. Said diesel can be selected either from diesels that fall within the specifications of diesel for transport according to standard EN 590:2009 or those for diesels that do not fall within said specifications. Said diesel may have a density, at 15° C., determined according to standard EN ISO 12185:1996/C1:2001, comprised between 780 kg/m³ and 845 kg/m³, preferably comprised between 800 kg/m³ and 840 kg/m³. Said diesel may have a flash point, according to standard EN ISO 2719:2002, greater than or equal to 55° C., preferably greater than or equal to 65° C. Said diesel may have a cetane number, determined according to standard EN ISO 5165:1998, or standard ASTM 06890:2008, greater than or equal to 47, preferably greater than or equal to 51. Diesels that can be used successfully for the purposes of the present disclosure may be all the known ones, possibly deriving from the mixture of diesel blends of different origins and compositions. Preferably the sulfur content of these diesel blends is comprised between 200 and 1 mg/kg, and even more preferably between 10 and 1 mg/kg. Typical diesels may be middle distillates, preferably having a boiling point comprised between 180 and 380° C., such as diesels from primary distillation, diesels from vacuum distillation, diesels from thermal or catalytic cracking, such as desulfurized diesel from fluid catalytic cracking, light cycle oil (LCO), diesels from a Fischer-Tropsch process or of synthetic origin. The term “diesel” also comprises so called green diesel and biodiesel blends and mixtures thereof with traditional refinery diesels.

For the purposes of the present description and following claims, the terms “gasoline” or “gasoline blend” mean a mixture prevalently comprising hydrocarbons such as, by way of example, paraffins, aromatic hydrocarbons, olefins and naphthenes, typically having from 3 to 12 carbon atoms, which can be used as fuel, characterized by an End Point (ASTM 086) not greater than 250° C., preferably not greater than 210° C., where the End Point means the temperature at which 100% by volume of said hydrocarbon mixture is distilled. Said gasoline may have a density comprised between 700 and 800, preferably between 720 and 775, kg/m³. Usable gasolines are those deriving from catalytic processes, preferably deriving from Fluid Catalytic Cracking (FCC) processes, from reforming processes, and mixtures thereof, according to what is generally known in the art. Preferably the sulfur content of these gasoline blends is comprised between 50 and 0.1 mg/kg, and even more preferably between 10 and 0.5 mg/kg. Unleaded gasolines are particularly preferred, which comprise mixtures of hydrocarbons having boiling points at atmospheric pressure in a relatively narrow temperature range, for example comprised between 25° C. and 225° ° C. Some gasolines may contain oxygenated compounds, such as alcohols (e.g., ethanol, propanol), or ethers (e.g., methyl-t-butyl-ether, MTBE). The gasolines may also comprise different additives such as detergents, anti-freeze agents, emulsion breakers, corrosion inhibitors, dyes, anti-depositing agents and octane boosters.

For the purposes of the present description and following claims, “from renewable sources” (e.g. “glycerin from renewable sources”) means compounds not obtained from fossil resources, such as crude oil, carbon, natural gas, oil sands, etc., but directly from plant biomass, algae, microorganisms or from the treatment of more complex compounds derived from said plant biomass, algae and microorganisms.

For the purposes of the present description and following claims, the term propanol, unless specified otherwise, means overall the set of isomers of propanol, i.e. 1-propanol, 2-propanol or both the isomers in mixture in any proportion with each other.

For the purposes of the present description and following claims, the term “conversion per pass” means the rate of conversion of the starting material, namely glycerol, calculated from input to output of the hydrogenation reactor.

According to a first aspect, the disclosure relates to a process for the conversion of glycerin, in particular glycerin from renewable sources (herein after “bio-glycerin”), to propanols that can be used as fuel components in bio-fuel mixtures.

The process of the disclosure comprises the following steps:
a) Hydrogenation of a glycerin phase with a Co—Cu—Mn—Mo based hydrogenation catalyst to give an effluent containing water and an organic mixture of more than 40 wt % of a mixture of ethanol, 1-propanol and 2-propanol and the rest being unreacted propanediols and glycerin, with traces of ethylene glycol;
b) Separating by distillation the ethanol, 1-propanol and 2-propanol mixture from the other components in the effluent of step a);
c) Optionally, recycling all or part of the unreacted propandiols and glycerine deriving from steps a) and/or b) to the hydrogenation step a).

The organic mixture in the effluent of step a) preferably contains:
at least 35 wt %, more preferably at least 40 wt %, of 1-propanol;
at least 5 wt %, more preferably at least 8 wt %, of a mixture of ethanol and 2-propanol;
less than 45 wt %, more preferably less than 37 wt %, of 1,2-propandiol;
less than 16 wt %, more preferably less than 7 wt %, of unreacted glycerin.

Depending on the reaction's conditions, the effluent mixture may also contain less than 8 wt %, more preferably less than 5 wt % of a mixture of other alcoholic components (ethylene glycol, 1,3-propanediol, acetol, traces of other alcohols) and acetone;

Glycerin can be any type of glycerin, preferably being or including bio-glycerin. The glycerin phase can consist of glycerin in a substantially pure form or of a glycerin/water mixture containing up to 25 wt %, preferably up to 20 wt %, more preferably up to 15 wt % water.

The glycerin in a substantially pure form preferably has a commercial purity grade of at least 98%.

Alternatively, when glycerin in a substantially pure form is to be used, it can be purified in advance from the excess salts and water that may be present in the event that it is derived from the transesterification of triglycerides. The crude glycerin can be subjected to a pre-treatment of purification to obtain glycerin with the desired degree of purity. Said purification can be performed, for example, through a process comprising two steps:
in the first step, the salts contained in the crude glycerin are removed, coming from the production of FAME, through treatment on acid exchange resins such as Amberlyst 15, Amberlyst 36, performing the removal preferably at temperatures comprised between 0° ° C. and 60° ° C. and even more preferably between 15° C. and 30° ° C., operating at atmospheric pressure;
in the second step the impurities present in the crude glycerin are removed, mainly comprising water with small amounts of methanol, through fractional distillation, until a glycerin content of at least 95−96% is obtained.

Further details related to the purification of glycerin are described, for example, in “PERP Report Glycerin conversion to propylene glycol 06/0784, March 2008”. The glycerin resulting from the steps described above may be used in the process according to the present disclosure without any further purification.

Step a) can be performed in glycerin as such or in the presence of a solvent. Possible solvents that can be used are for example the same reaction products propanols and propanediols, preferably in the same proportions desired for the product of the hydrogenation reaction. Another solvent or co-solvent may be water, which however constitutes a reaction product and is always present in the mixture exiting from step (a).

The hydrogenation catalyst is preferably a carrier-free hydrogenation catalyst which, in the calcined, non-reduced state, contains from 40 to 70% by weight, preferably 64−68% by weight, cobalt (in the form of Co₃O₄), from 13 to 22% by weight, preferably 18−20.5% by weight, copper (as CuO), from 3 to 8% by weight, preferably 6.6-7.8% by weight, manganese (as Mn₃O₄), from 0.1 to 5% by weight, preferably 2.5−3.5% by weight, phosphorous (as H₃PO₄), from 0.5 to 5% by weight, preferably 3−4% by weight, molybdenum (as MoO₃), and from 0 to 10% by weight of an alkali metal oxide.

The hydrogenation is conducted at a temperature between 220° C. and 270° ° C., preferably between 240° C. and 260° C., more preferably of about 250° C., and at a pressure between 130 and 170 bar, preferably between 140 and 160 bar, more preferably of about 150 bar.

An important parameter in the present process is the LHSV. The LHSV (Liquid Hourly Space Velocity) by mass—defined as the ratio of the fresh glycerin phase feed (in kg/hr) to the catalyst weight (in kg)-is comprised preferably between 0.15 and 2 hr-1, more preferably between 0.15 and 1.0 hr-1, even more preferably between 0.2 and 0.7 hr-1, most preferably between 0.23 and 0.5 hr-1.

The hydrogenation catalyst is obtainable according to the method described in U.S. Pat. No. 5,107,018, that is herein enclosed by reference.

According to this method, salts of cobalt, copper and manganese, and phosphoric acid are mixed in aqueous solutions, precipitated as metal salts in a two-stage precipitation by firstly bringing the solution to a pH-value of 8 by addition of an alkali metal carbonate solution at a temperature between 30° C. and 70° C., then adjusting to a pH-value of less than 7.5 by addition of further metal salt solution. The precipitate is collected by filtration or centrifugation, then calcined into the according oxides at a temperature between 400° ° C. and 600° C. The recovered material is cooled after the calcination, post-washed if necessary, then impregnated with a salt of molybdic acid and fixed to the mass by acid treatment with molybdic acid, then formed, dried and activated by reduction with hydrogen.

In step a), the conversion of glycerin per pass to products is more than 70%, preferably more than 80%, more preferably more than 90%.

The process according to the present disclosure may be performed using conventional pressure equipment known to a person skilled in the art.

More particularly, step a) can be conducted as follows.

The glycerol is firstly compressed and heated to reach the operative conditions for the reaction. The heating process can be performed by recovering the heat of the effluent from the reactor.

The reactor is a trickle bed, with a single catalytic bed or multiple beds interlaced by gaseous quenches, filled with the catalyst as defined above.

The gas stream, containing mainly hydrogen, is mixed with the feed stream before entering the reactor. The mixed stream enters the reactor, where it is contacted with the catalyst. Here, the conversion of glycerol to 1,2-propanediol, followed by the conversion of the latter into 1-propanol and iso-propanol are obtained. As co-products of the hydrogenation, ethanol, 1,3-propanediol, ethylene glycol, traces of acetol and acetone and a small quantity of gases such as methane, propane and ethane may be produced. Water is also produced as a by-product.

Downstream the reactor and upstream step b), a series of flashing units with decreasing temperature and pressure, to separate the gaseous products from the liquids, are provided.

As the hydrogenation is strongly exothermic, an increase of temperature is expected in the catalytic volume. To keep the temperature within acceptable limits for the stability of the catalyst, according to certain embodiments, an amount of the flashed liquid effluent of the flashing unit is recycled and mixed with the feed. Another embodiment provides for the control of exothermicity by the introduction of cold gas in the catalytic volume, which can be divided in two or more beds with intermediate quenches.

The flashed gas stream exiting the above described flashing unit, on the other hand, provides the gas recycle loop and delivers duty to pre-heat both the hydrogen and the feed upstream step b).

As the off-gases containing hydrogen could generate issues in the distillation section, a series of additional flashing units (e.g., three flashing units) is placed downstream the reactor, so that off gasses containing excess hydrogen, methane and traces of propane and ethane are separated from the liquid phase.

Step b) of separation by distillation of ethanol, 1-propanol and 2-propanol (alcohol phase) from the other components (diol phase) in the effluent of step a) comprises the following stages:
i) Distillation of the effluent by head separating the alcohol phase and water from the diol phase and the unreacted glycerin;
ii) Distillation of the alcohol phase and water from stage i) by head separating the alcohol phase from water by means of extractive distillation with ethylene glycol as entrainer;
iii) Optionally, distillation of ethylene glycol and water from stage ii) by bottom recovering ethylene glycol.

Step b) can be performed at atmospheric pressure, at a slight overpressure or under vacuum.

In stage ii), the ratio between the ethylene glycol feed rate (in Kmol/hr) and alcohol phase/water feed rate (in Kmol/hr) is comprised between 2 and 3.5. When calculated on the basis of feed rates expressed in Kg/hr, this ratio is comprised between 0.5 and 6.5.

The distillation can be performed through fractional distillation, continuously or discontinuously, preferably continuously, using, for example, an appropriately sized fractionating column. Each of the stages i), ii) and iii) of the separation step (b) can be performed through two or more distillation columns, placed in series with each other. The boiling points and phase diagrams of the different compounds and mixtures thereof are well known and adapted to allow separation as required.

The columns used may be made of stainless steel or other suitable materials according to what is known in the state of the art.

According to a preferred embodiment of the present disclosure, the unreacted glycerin separated in step (b) is recycled to step (a), together with ethylene glycol and propandiols.

In a preferred embodiment, the distillation section consists of three distillation columns.

The first distillation column separates the mixture composed by propanol, iso-propanol, ethanol and water from the other reactor effluent components. The heaviest components, mainly 1,2 propanediol, 1,3 propanediol and ethylene glycol along with unreacted glycerol, are removed from the bottom.

The second column is an extractive distillation unit which is able to separate water and alcohols in the azeotropic mixture. To do so, the column requires ethylene glycol as a solvent capable of entraining water. As a result, high purity propanol, iso-propanol and ethanol can be recovered from the top, while the solvent and the water are removed from the lowest stage and can be sent to the entrainer recovery column.

This third column performs the regeneration of the ethylene glycol, which is recycled from the bottom of the column to the extractive unit, while water is separated on top of the column.

For the uses of 1-propanol as a mixture component of gasolines, the possible presence of ethanol in the mixture does not represent any drawback and can also be successfully used as a component for gasolines, without any further separation.

When recycling step c) is performed, the Combined Feed Ratio (CFR, given by the ratio between combined fresh and recycle feed/fresh feed) is preferably less than 20 and more than 5. A CFR lower than 5 would not allow a sufficient control of the temperature in the hydrogenation reactor (the reaction is exothermic) while a CFR greater than 20 would imply a considerable increase in the plant CAPex & OPex costs

### EXAMPLE 1

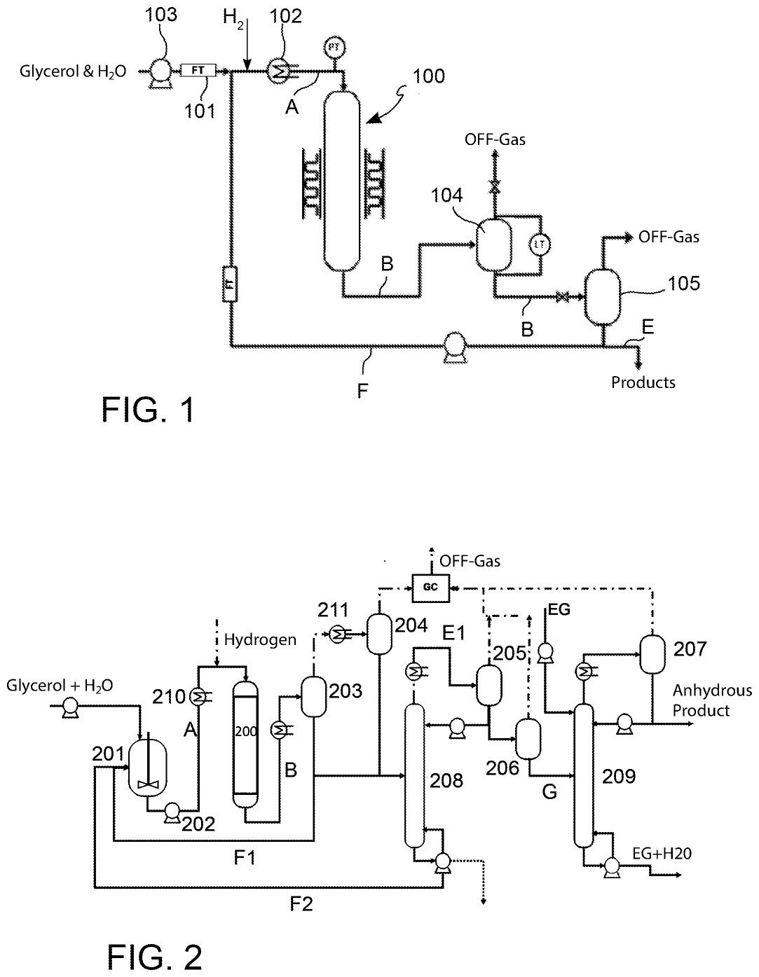

FIG. 1 shows a bench scale simplified process scheme.

A 1″ fixed bed reactor 100 was charged with 183 grams of hydrogenation catalyst C as defined above. A mixture of glycerin-water at a feed rate of 80 g/hr, of which 68 g/h of glycerin (feed A), was fed long with a feed of hydrogen (150 Nl/hr). The mixture A was fed, via a pump 103 and through a heat exchanger 102, to the fixed bed reactor 100 at 240° C. and 150 bars.

The liquid space hourly velocity (LSHV) was 0.44 h⁻¹ based on fresh feed, and a Liquid Recycle of 1.8 kg/hr and a Combined Feed Ratio (CFR) of 22,5 were applied.

Two separation vessels 104, 105 were positioned downstream the reactor 100 to eliminate from the reactor effluent B the gaseous products (OFF-Gas), among which unreacted hydrogen.

The final product mixture E was recovered from the bottom of the second separation vessel 105. Part of this product mixture is recycled (stream F) to the feed stream A.

The composition (wt %) of the liquid effluent B from the reactor 100 by analysis is reported as follow:
Aqueous Mixture: 37%
Water: 100%
Organic Mixture: 73%
Ethanol+Iso-Propanol=8.0%
n-Propanol=40.0%
1,2 Propandiol=35.0%
Ethylene Glycol+1,3 Propandiol=2.0%
Glycerol=15.0%.

### EXAMPLE 2

FIG. 2 represents a plant that processes 200 to 500 cc/h of glycerol at 85 wt % purity using 1.44 kg of catalyst C loaded in the tubular reactor 200 with an internal diameter of 55 mm. The feed stream A is mixed with pure hydrogen before entering the reactor 200. The plant includes a mixing vessel 201 to accommodate the combined feed made of fresh glycerol/water and recycled streams F1, F2 (see below) to the reactor. The feed stream A is circulated by pump 202 and is heated through a heat exchanger 210.

The reactor effluent B passes through a first separation vessel 203 operated at high temperature to separate the off gasses; the gaseous phase from 203 is sent to another separation vessel 204 while being previously subjected to temperature reduction in the heat exchanger 211 to recover some of the vaporized products, in order not to lost them in the off-gasses. Part of the products exiting the bottom of the first separation vessel 203 is recycled to the mixing vessel 201, while the rest of the products is sent to a low-pressure distillation column 208 together with the liquid phase from the separation vessel 204. In the distillation column 208, the product fraction (propanols and ethanol) and water (stream E1) are separated at the column head. The bottom stream of the distillation column 208, which contains unreacted glycerol and propanediols, can be recycled to the mixing vessel 201 if a complete conversion of the reagents is desired.

The stream E1 is fed to two further separation vessels 205, 206 to further separate off gases from the liquid phase (stream G). The stream G containing the products and water, is sent to a second distillation column 209, which performs the dehydration of the product using ethylene glycol (EG) as entrainer. In the distillation column 209, the product fraction (propanols and ethanol) is separated at the column head with a water content lower than 2500 ppmw and, after being passed through a fourth separation vessel 207 to finally eliminated off-gases, is recovered. The EG and water stream from the bottom is eventually regenerated offline by heating the mixture.

The plant worked with a basic LHSV of about 0.3 h⁻¹. The inlet temperature in the hydrogenation reactor 200 was 250° C. The reactor pressure was 150 bar. The following table summarizes the main parameters of the plant during a standard test:

| | |
|---|---|
| Inlet Reactor T [° C.] | 250 |
| Catalyst load [kg] | 1.44 |
| Inlet Reactor P [bar] | 150 |
| Fresh feed [kg/h] | 0.41 |
| Stream F1 feed [kg/h] | 2.415 |
| Stream F2 feed [kg/h] | 0.4 |
| LHSV [h⁻¹] | 0.28 |
| Combined feed ratio | 7.9 |
| (CFR) by mass | |
| Hydrogen [NL/h] | 450 |

The reactor liquid effluent B composition (wt %) by analysis is reported as follow:
Aqueous Mixture: 40%
Water: 100%
Organic Mixture: 60%
Ethanol+Iso-Propanol=9.0%
n-Propanol=42.0%
1,2 Propandiol=41.0%
Ethylene Glycol+1,3 Propandiol=2.0%
Glycerol=6.0%

The reactor liquid effluent B is then fed to the distillation section. The final product stream leaving the head of column 209 has the following composition:
Ethanol=8.0%
Iso-Propanol=10.0%
n-Propanol=82%
Water: ≤ 2500 ppmw.

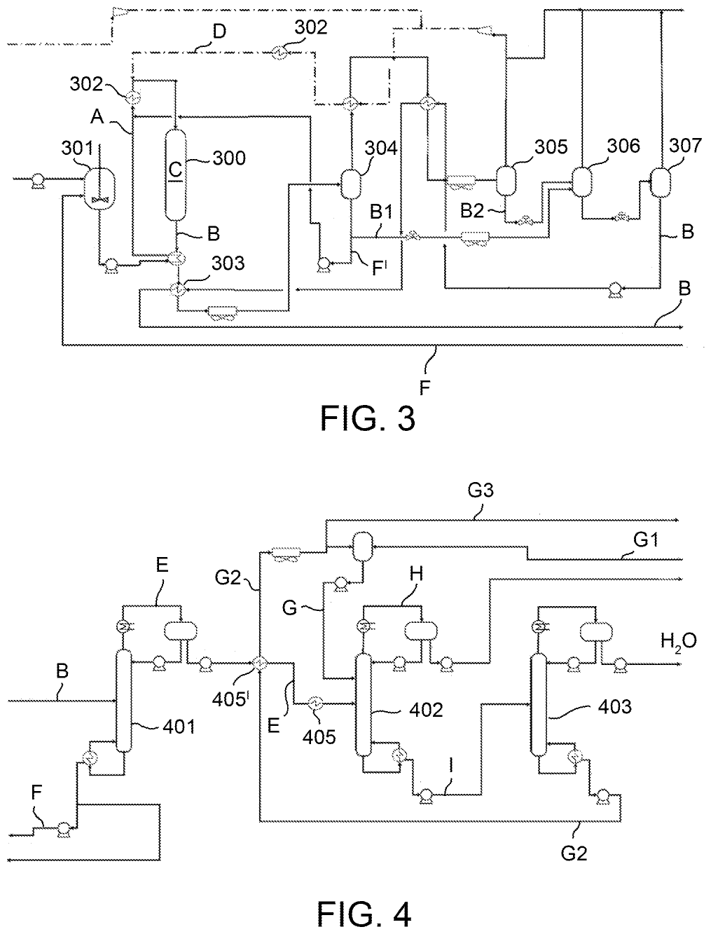

FIGS. 3 and 4 illustrate another example of a plant for performing the industrial process of the disclosure.

Specifically, FIG. 3 shows the section of the plant for performing the hydrogenation reaction (step a), while FIG. 4 shows the specific section of the plant for performing the separation step b).

The fresh feed of glycerol (pure or in admixture with water) is conveyed in a mixing vessel 301 wherein it is admixed with a recycled stream F containing ethylene glycol+propandiols (coming from distillation column 401, see FIG. 4). The feed stream A is compressed and heated (through a heat exchanger 302) at the hydrogenation conditions (see description below) and it is added with a stream D of hydrogen, also heated and compressed at the needed temperature and pressure.

The stream A is sent to a hydrogenation reactor 300 filled with the hydrogenation catalyst C. The effluent B is cooled in a heat exchanger 303, then is sent to a first separation vessel 304, wherein the liquid effluent B1 is recovered from the bottom and it is partly recycled (stream F′) to the feed stream A. The gaseous products, together with some liquid product stripped off, is sent to a second separation vessel 305 which still separates the gaseous products (head effluents) from the liquid products (bottom effluent B2).

Liquid products streams B1, B2 are sent to a third separation vessel 306, then to a fourth separation vessel 307, wherein the gaseous products are definitely separated from the liquid products (stream B) that are sent to the step b) of separation by distillation.

The gaseous products recovered from the second separation vessels 305, mainly containing hydrogen, are partly recycled and added to the hydrogen fresh feed sent to the reactors 300, while the gaseous products of the third and fourth separation vessels 306, 307 and the remaining portion of the gaseous effluent of the second separation vessel 305 are recovered as off gases.

With reference to FIG. 4, the step b) of separation by distillation comprises a first distillation column 401 wherein the liquid products coming from step a) (stream B) are fed. In the distillation column 401, the alcohol fraction and water (stream E) are separated at the head, while the mixture of ethylene glycol, propandiols and unreacted glycerin are recovered from the bottom and at least in part recycled to the hydrogenation reactors 300 (stream F).

The alcohol fraction—containing propanols and ethanol—and water (stream E) are partly recycled to the head of distillation column 401, the rest being fed to an intermediate portion of a second distillation column 402 after having been heated through a heat exchanger 405, preferably a shell and tube heat exchanger. At the top of the same column 402 it is fed ethylene glycol as an entrainer (stream G). The second distillation column 402 separates at the head the alcohol fraction deprived of water (stream H) and at the bottom a mixture of ethylene glycol and water (stream I) that is fed to a third distillation column 403.

The third distillation column 403 is a recovery column for ethylene glycol. In column 403, water is distilled off, while substantially anhydrous ethylene glycol is recovered at the bottom (stream G2) and it is added to fresh ethylene glycol (stream G1) to constitute stream G as a feed for the second distillation column 402. Since stream G2 exits the third column 403 at a high temperature (about 200° C.), collected stream G must be cooled in the heat exchangers 405′ before being fed in the second column 402.

The stream B of products coming from hydrogenation step a) is preferably heated at a temperature of 140−150° C. before feeding in the first distillation column 401. This column is preferably a 11-stage column and operates at a slight overpressure, for example about 1.5 atm.

The second distillation column 402 is preferably a 60-stage column. Stream E is preferably fed at a temperature comprised between 140° C.-160° C. or about 150° C., while ethylene glycol (stream G) is fed at a temperature selected in the range of from room temperature to 150° C. While operating at a temperature between room temperature and 40° C. the water absorption is maximised. On the other hand, the 120-140° C. temperature range is preferred if an improved heat recovery and energy saving is desired.

The alcohol fraction (stream H) recovered at the head of the second distillation column 402 typically contain less than 1 mol %, preferably less than 0.5 mol % of a mixture of ethylene glycol and propandiols and less than 3000 ppm, preferably less than 2600 ppm of water.

In an example, the alcohol fraction of stream H has the following composition:
1-propanol 27.4 mol %
2-propanol 46.6 mol %
Ethanol/methanol 24.7 mol %.

The third distillation column 403 can be a 20-stage column, wherein the mixture ethylene glycol/water is fed at an intermediate portion and at a temperature between 155° ° C. and 170° C.

The above described plant is just an example and it can be modified according to specific need.

For example, more than one hydrogenation reactor 300 can be provided. If at least two hydrogenation reactors 300 are provided, they can be put in parallel or in series.

The number of separation vessels 304, 305, 306, 307 can be calculated in view of the reaction conditions and of the size and productivity of the plant.

When the conversion of glycerol is very high or in other operative necessities, the recycled stream F can also be omitted.

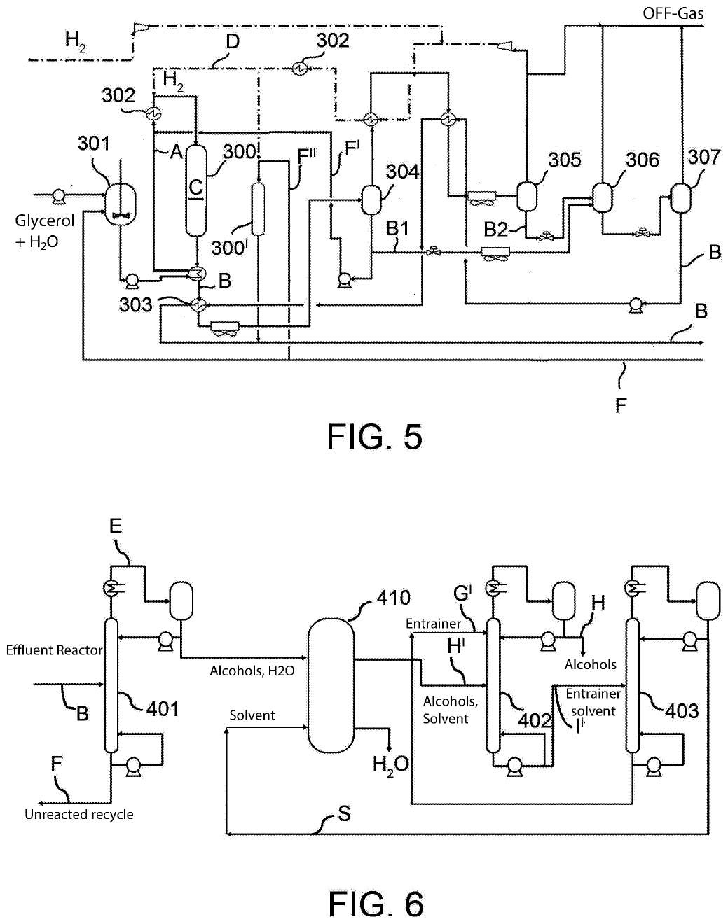

FIG. 5 depicts a variant of the plant described in connection with FIG. 3. The process design is the same as that described above in connection with FIG. 3, but for the presence of a second hydrogenation reactor 300′, similar to reactor 200, that acts as a finisher for the reaction. The finishing reactor 300′ contains a catalyst mass 3 to 5 times lower than the main reactor 300. The finishing reactor 300′ can receive a stream F″ which is part of the stream F from the bottom of the first distillation column 401 (see FIG. 4), that is concentrated in reactive species, namely propanediol and residual glycerol. In the finishing reactor 300′ it is possible to achieve the complete conversion of glycerol and an almost complete conversion of propanediols. The effluent from hydrogenation finishing reactor 300′ is then sent to the first distillation column 401 to separate the alcohol fraction and water formed in the reaction.

The advantage of the alternative configuration with a secondary reactor is the possibility of performing the reaction on a very concentrated stream, thus removing or reducing the amount of unreacted species to be recycled to the main reactor. The disadvantages are the cost associated with the installation of another reactor, which includes the design of a more effective way of controlling the exothermicity of the reaction (i.e. gaseous quenches) when operating with an inlet stream without water, that acts as a diluent and help in controlling the increase of temperature.

FIG. 6 depicts a variant of the distillation section shown in FIG. 4, that is provided in order to improve the utilities consumption and the amount of entrainer needed for the purification of alcohols.

The distillation section comprises a first distillation column 401 wherein the liquid products coming from step a) (stream B) are fed. In the distillation column 401, the alcohol fraction and water (stream E) are separated at the head, while the mixture of ethylene glycol, propandiols and unreacted glycerin are recovered from the bottom and at least in part recycled to the hydrogenation reactors 300 (stream F).

The alcohol fraction—containing propanols and ethanol—and water (stream E) are partly recycled to the head of distillation column 401, the rest being fed to a liquid-liquid extraction vessel 410, wherein stream E is put into contact with a treatment solvent preferably selected from toluene, hexane, cyclohexane, methylcyclohexane, heptane, isooctane and DIPE. Water is removed from the bottom of the vessel 410, while the alcohol fraction and the treatment solvent (stream H′) are separated at the top of the vessel 410.

The stream H′ is fed to a second distillation column 402. At the top of the same column 402 it is fed an entrainer (stream G′), for example ethylene glycol. The second distillation column 402 (extractive distillation column) separates at the head the substantially anhydrous alcohol fraction (mainly ethanol, 1-propanol and 2-propanol, stream H) and at the bottom a mixture of entrainer and treatment solvent (stream I′) that is fed to a third distillation column 403.

The third distillation column 403 separates at the bottom the entrainer (stream G′) which is then fed to the second distillation column 402, while the treatment solvent is recovered at the top of column 403 and sent to the liquid-liquid extraction vessel 410 (stream S).

The above described variant allows to minimize the heat and energy consumption in the distillation stages.

1. A process for the conversion of glycerin to propanols, the process including the following steps:
a) Hydrogenating a glycerin phase with a Co—Cu—Mn—Mo based hydrogenation catalyst to give an effluent containing water and an organic mixture of more than 40 wt % of a mixture of ethanol, 1-propanol and 2-propanol and the rest being unreacted propanediols and glycerin, with traces of ethylene glycol;
b) Separating by mainly distillation the ethanol, 1-propanol and 2-propanol mixture from the other components in the effluent of step a);
c) Optionally, recycling all or part of the unreacted propandiols and glycerine deriving from steps a) and/or b) to the hydrogenation step a).

2. The process of claim 1, wherein the organic mixture in the effluent of step a) contains:
at least 35 wt % of 1-propanol;
at least 5 wt % of a mixture of ethanol and 2-propanol;
less than 45 wt % of 1,2-propandiol;
less than 16 wt % of unreacted glycerin.

3. The process of claim 1, wherein the glycerin phase comprises glycerin in a substantially pure form or of a glycerin/water mixture containing up to 25 wt % water.

4. The process according to claim 1, wherein the hydrogenation catalyst is a carrier-free hydrogenation catalyst which, in the calcined, non-reduced state, contains from 40 to 70% by weight, cobalt in the form of Co₃O₄, from 13 to 22% by weight, copper as Cu), from 3 to 8% by weight, manganese as Mn₃O₄, from 0.1 to 5% by weight, phosphorous as H₃PO₄, from 0.5 to 5% by weight, molybdenum as MoO₃, and from 0 to 10% by weight of an alkali metal oxide.

5. The process according to claim 1, wherein the hydrogenation is conducted at a temperature between 220° C. and 270° C., and at a pressure between 130 and 170 bar.

6. The process according to claim 1, wherein the LHSV by mass, defined as the ratio of the fresh glycerin phase feed in kg/hr to the catalyst weight in kg, is comprised between 0.15 and 2 hr.

7. The process according to claim 1, wherein, in step a), the conversion of glycerin per pass to products is more than 70%.

8. The process according to claim 1, wherein step b) of separation by distillation of ethanol, 1-propanol and 2-propanol as an alcohol phase from the other components as a diol phase in the effluent of step a) comprises the following stages:
i) Distillation of the effluent by head separating the alcohol phase and water from the diol phase and the unreacted glycerin;
ii) Distillation of the alcohol phase and water from stage i) by head separating the alcohol phase from water by means of extractive distillation with ethylene glycol as entrainer;
i) Optionally, distillation of ethylene glycol and water from stage ii) by bottom recovering ethylene glycol.

9. The process according to claim 8, wherein in stage ii), the ratio between the ethylene glycol feed rate in Kmol/hr and alcohol phase/water feed rate in Kmol/hr is comprised between 2 and 3.5; or the ratio between the ethylene glycol feed rate in Kg/hr and alcohol phase/water feed rate in Kg/hr is comprised between 0.5 and 6.5.

10. The process according to claim 8, wherein, when recycling step c) is performed, the Combined Feed Ratio (CFR, given by the ratio between combined fresh and recycle feed/fresh feed) is less than 20 and more than 5.

11. The process according to claim 8, wherein step b) can be performed at atmospheric pressure, at a slight overpressure or under vacuum.

12. The process according to claim 1, wherein step b) of separation by distillation of ethanol, 1-propanol and 2-propanol as an alcohol phase from the other components as a diol phase in the effluent of step a) comprises the following stages:
i) Distillation of the effluent by head separating the alcohol phase and water from the diol phase and the unreacted glycerin;
ii) Treatment by liquid-liquid extraction of the alcohol phase and water with a treatment solvent, selected from the group consisting of toluene, hexane, cyclohexane, methylcyclohexane, heptane, isooctane and DIPE, and head separating the alcohol fraction and the treating solvent from the water;
iii) Distillation of the alcohol phase and the treatment solvent from stage ii) by head separating the alcohol phase from the treating solvent by means of extractive distillation with an entrainer;
iv) Optionally, distillation of the entrainer and the treatment solvent from stage iii) by top recovering the treating solvent.

13. A plant for actuating the process of claim 1, comprising:
at least one hydrogenation reactor filled with the hydrogenation catalyst (C);
at least one first distillation column configured for separating the mixture composed by propanol, iso-propanol, ethanol and water coming from the at least one hydrogenation reactor from the other reactor effluent components, wherein the heaviest components, mainly 1,2 propanediol, 1,3 propanediol and ethylene glycol along with unreacted glycerol, are removed from the bottom;
at least one second distillation column configured for extractive distillation to separate water and ethylene glycol as a an entrainer solvent from the bottom and high purity propanol, iso-propanol and ethanol from the top;
optionally, at least one third distillation column configured to separated ethylene glycol from the bottom and water from the top.

14. A plant for actuating the process of claim 12, comprising:
at least one hydrogenation reactor filled with the hydrogenation catalyst;
at least one first distillation column configured for separating the mixture composed by propanol, iso-propanol, ethanol and water coming from the at least one hydrogenation reactor from the other reactor effluent components, wherein the heaviest components, mainly 1,2 propanediol, 1,3 propanediol and ethylene glycol along with unreacted glycerol, are removed from the bottom;
at least one liquid-liquid extraction vessel configured for bottom separating, by extraction with a treatment solvent, water from an alcoholic fraction and the treatment solvent;
at least one second distillation column configured for extractive distillation to separate the treatment solvent and an entrainer from the bottom and high purity alcohol fraction from the top;
optionally, at least one third distillation column configured to separate the entrainer from the bottom and the treatment solvent from the top.

15. The plant according to claim 13, comprising a first hydrogenation reactor and a second hydrogenation reactor, wherein:
the first hydrogenation reactor receives a mixture of fresh glycerin feed and a first part of recycled products feed from the first distillation column, and
the second hydrogenation reactor receives a second part of recycled products feed from the first distillation column.
